(19) United States
(12) Patent Application Publication
(10) Pub. No.: US 2003/0078523 A1
(43) Pub. Date: Apr. 24, 2003

(54) Medical device

(75) Inventors: Michael Burkhardt, Muhlacker (DE); Reinhard Pomper, Knittlingen (DE); Werner Krauss, Knittlingen (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: Richard Wolf GmbH

(21) Appl. No.: 10/246,317

(22) Filed: Sep. 17, 2002

(30) Foreign Application Priority Data
Sep. 17, 2001 (DE) 101 45 852.5

(51) Int. Cl. A61B 5/05

(52) U.S. Cl. 601/2; 601/4; 600/411

(57) ABSTRACT

The invention relates to a medical device with a picture-providing diagnostic X-ray system and a therapy source, in particular a lithotriptor. For improving the accessibility of the device on examination, according to the invention it is envisaged for the therapy source to be arranged on a guide arm whose one end is translatorily movable relative to the X-ray system into a displacement direction, and for the guide arm to be pivotably arranged about a rotational axis.

### BACKGROUND OF THE INVENTION

The device relates to a medical device with a picture-providing diagnostic X-ray system and to a therapy source.

With devices of the above mentioned type one may for example remove body stones in a non-evasive manner. In order to be able to destroy a body stone the therapy source, specifically the lithotriptors, and the X-ray system must be aligned relative to one another. The focusses of both apparatus must be brought to converge since the stone to be destroyed must be arranged in the target cross of the X-ray system.

In order to ensure the coordination of the focusses in the target cross of the X-ray system, it is known from DE 39 16 093 A 1 to apply a device with which the therapy source (the electroacoustic transducer for the ultrasound emission) and the X-ray system (the location means for the stone to be destroyed) are rigidly connected to one another. With this device it is of a disadvantage that there is not the possibility of being able to use the therapy source on the one hand and the X-ray system on the other hand also independently of one another in order thus to render the high cost of purchase commensurate when the applications become more comprehensive.

From DE 43 00 740 C1 and U.S. Pat. No. 5,065,741 there are known devices with which the therapy source may be set next to an X-ray system. The allocation of the X-ray system to the therapy source however here requires the necessary corresponding adjusting work which is mostly time consuming and thus burdens the patients. It continues to be of a disadvantage with these devices that the adjusting work becomes necessary again for each movement of the apparatus which were previously aligned to one another, in order to avoid an erroneous focussings on account of maladjustment.

From DE 197 46 956 C2 there is further known an X-ray system with a therapy source, wherein with this device the X-ray system equipped with a C-shaped X-ray arc may be axially displaced with respect to the therapy source and well as pivoted about a horizontal axis. Both device parts—thus the X-ray system on the one hand and the therapy source on the other hand—are releasably connected to one another via a mechanical coupling. In DE0 739 609 B 1 there is disclosed a lithotripsy combination, consisting of an X-ray system and therapy source, with which the X-ray system may be pivoted about a horizontal rotational axis into a parked position in order to be able to visually examine a patient without hindrance. With the solutions according to the two last mentioned documents it has been found to be disadvantageous that the measures provided in order to move the therapy source away from the patient and thus to have an unhindered access to him are unsatisfactory.

### BRIEF SUMMARY OF THE INVENTION

It is therefore the object of the invention to further develop a device of the known type such that it is possible, when required, to be able to exactly align the therapy focus of the therapy source and the target cross of an X-ray system quickly and exactly to one another. Furthermore at the same time it is to be ensured that the therapy source at any time may be removed out of the position aligned onto the area which is to undergo therapy, wherein subsequently and when required, the positioning onto the body to be treated is to be possible without new, complicated adjustment work. In the position in which the therapy source is removed from the aligned position, the operating physician is to be given adequate space for carrying out visual examinations.

The solution of this object by the invention is characterised in that the therapy source is arranged on a guide arm whose one end is translatorily movable relative to the X-ray system into a displacement direction, wherein the guide arm is pivotably arranged about a rotational axis.

Accordingly the guide arm on which the therapy apparatus is directly or indirectly arranged, is thus translatorily displaceable with respect to the X-ray system as well as pivotable about a rotational axis. With this the therapy source may be quickly removed from the therapy region in a particularly favourable manner in order to have a free access to the patient. Subsequently the therapy apparatus may just as quickly again be placed into the exact position in order to continue the treatment with it. Preferably the translatory displacement direction lies in a horizontal plane, whilst the rotational axis is arranged vertically.

The quick removal and renewed positioning of the therapy source is simplified when on the guide arm and/or on the X-ray system there are arranged means which with the translatory displacement of the guide arm relative to the X-ray system and the pivot angle of the therapy source relative to the X-ray system may be measured. By way of such measuring means a quick and exact setting of the guide arm may be accomplished.

Advantageously the one end of the guide arm is arranged on a carriage which may move translatorily on the X-ray system. With this one achieves a simple design embodiment.

The picture-providing diagnostic X-ray system may comprise an X-ray source and an X-ray receiver which are connected to one another via a connection element. At the same time a C-shaped arc ora U-shaped arc are advantageously applied as a connection element.

For the exact angle setting of the therapy source a pivot rail may be arranged between this and the guide arm, preferably at the end of the guide arm. For permitting a lateral adjustment of the X-ray system which is known per se, one may envisage distancing the rotational axis from the middle axis of the X-ray system sufficiently large.

The pivoting and translatory adjustment of the therapy source according to a further development of the invention is accomplished by movement means which move the guide arm on account of a control unit.

By way of the axial adjustment of the carriage arranged on the X-ray system by pivoting the therapy source, this when required may be quickly removed from the treatment area of the patient if the patient is to be brought into another position or the therapy source is to be brought into another position on the patient.

The physician often may only ascertain himself by testing various positions, which entry direction is the best for the therapy source. The therapy source however on account of the contour of the body of the patient may not simply be rotated around the patient without having to move the patient himself out of the therapy position. This however has the consequence of a repositioning of the patient. By way of the possibility of the translatory displacement of the therapy source in combination with its pivoting according to the present concept, the therapy source may now be removed from the patient with an unchanged position of the patient, newly aligned and again be brought into the exact position.

If there exists the desire to use the X-ray unit exclusively for diagnosis purposes, thus without a therapy source, then the guide arm together with the pivot rail and the therapy source arranged thereon may be completely pivoted out of the application region of the X-ray system.

If for example after an X-ray control has been effected with the therapy source pivoted away, the treatment of the patient is continued by destruction of body stones, it is merely necessary to pivot the guide arm back into the original position and by way of the axially displaceable carriage to lead the guide arm together with the therapy source to the patient, and specifically without the requirement of a new time-consuming adjustment.

A further advantage of the possibility of the displacement and pivoting of the therapy source relative to the X-ray system lies in the following. The therapy source may in certain therapy directions be disadvantageously located above or below in the irradiation field of the X-ray system. With the suggested formation for location during the irradiation there now exists the possibility of moving the therapy source out of the irradiation field into a parking position and subsequently of moving the therapy source quickly again into the correct treatment position.

### BRIEF DESCRIPTION OF THE DRAWINGS

In the drawings there are shown embodiment examples of the invention. There are shown in:

FIG. 1 the lateral view of a medical device with an X-ray system and a therapy source in the position for treatment of a patient,

FIG. 2a the device corresponding to the representation of FIG. 1,

FIG. 2b a plan view according to section 1-1 according to FIG. 2,

FIG. 2c the section according to FIG. 2b, with which apart from the treatment position of the therapy source, its parking position is also shown,

FIG. 3a the representation according to FIG. 2a, with the therapy source in the parking position,

FIG. 3b the plan view according to the section 11-11 according to FIG. 3a,

FIG. 4 the X-ray system and therapy source in the treatment position,

FIG. 5 corresponding to FIG. 4, the X-ray system and the translatorily traversed therapy source,

FIG. 6a an alternative embodiment form according to FIG. 2a,

FIG. 6b the representation according to FIG. 2c of the formation according to FIG. 6a with the section 111-111 according to FIG. 6a,

FIG. 7a further alternative embodiment form corresponding to FIG. 2a,

FIG. 8a, FIG. 8b and FIG. 8c three various positions of the therapy source on its pivot rail,

FIG. 9 schematically, the lateral adjustability of the X-ray system about the therapy focus and

FIG. 10 schematically, the orbital adjustability of the X-ray system about the therapy focus.

### DETAILED DESCRIPTION OF THE INVENTION

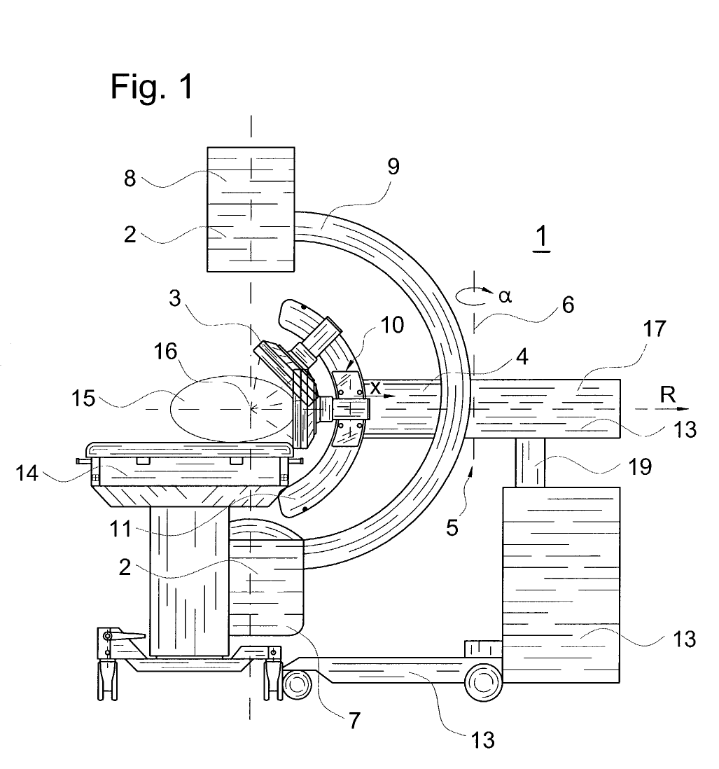

In FIG. 1 a device 1 according to the invention is to be seen in the lateral view, which serves for the destruction of body stones. It comprises a picture-providing X-ray system 2 which consists of the X-ray irradiation source 7 and an X-ray irradiation receiver8 which are connected to one another via a connection element 9 which is formed C-shaped. Furthermore the medical device 1 comprises a therapy source 3 in the form of a lithotriptor which may produce ultrasound waves for destroying the body stones. The X-ray system 2 and the lithotriptor 3 are arranged on a common base frame 13. The whole arrangement may be lifted or lowered via a lift arm 19.

A schematically indicated patient 15 lies on a patient table 14. So that the destruction of the body stones may be effected by way of the therapy souce under X-ray, the focus of the therapy source 3 and the focus of the X-ray system 2 must be brought to coincide. The focus of both apparatus is indicated at 16.

The therapy source 3 is arranged on a pivot rail by way of which it may be exactly aligned onto the focus 16. The pivot rail 11 is for its part connected to one end 10 of a guide arm 4 and is axially displaceable on this. The axial adjustment of the pivot rail may also be achieved in a manner different from this, if one designs the guide arm as a telescope arm. As is schematically to be seen in FIG. 1, the other end 5 of the guide arm 4 may be displaced translatorily in a displacement direction R by the amount x as well as pivoted about a vertical rotational axis 6 about a pivot angle 6. At the same time the end 5 of the guide arm 4 is connected to a carrier arc 17 of the X-ray system in a pivotably movable manner in a rotational joint which is not shown in more detail.

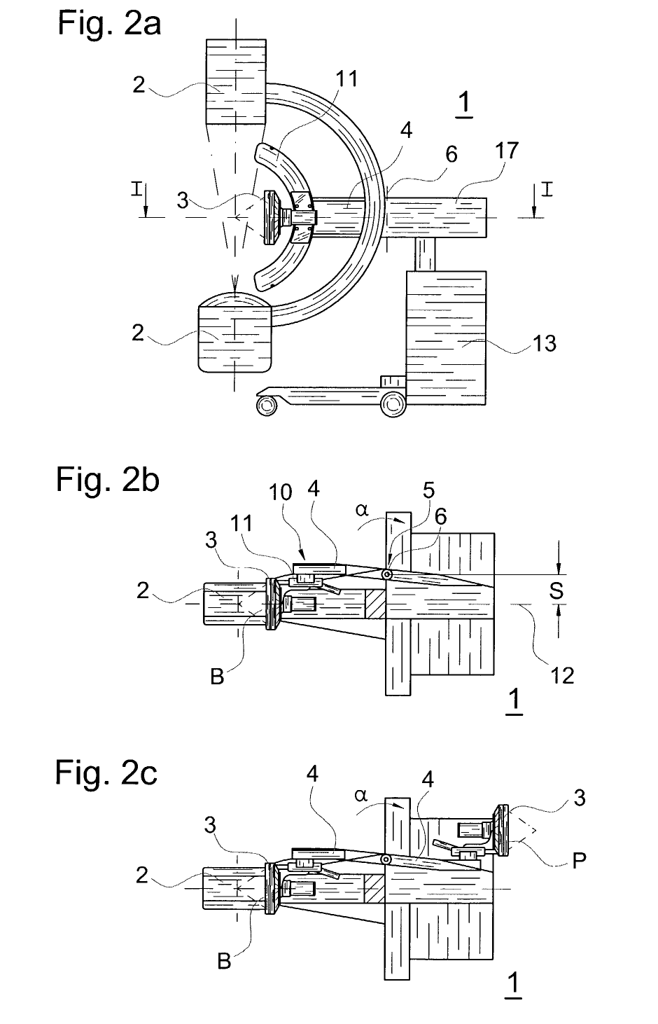

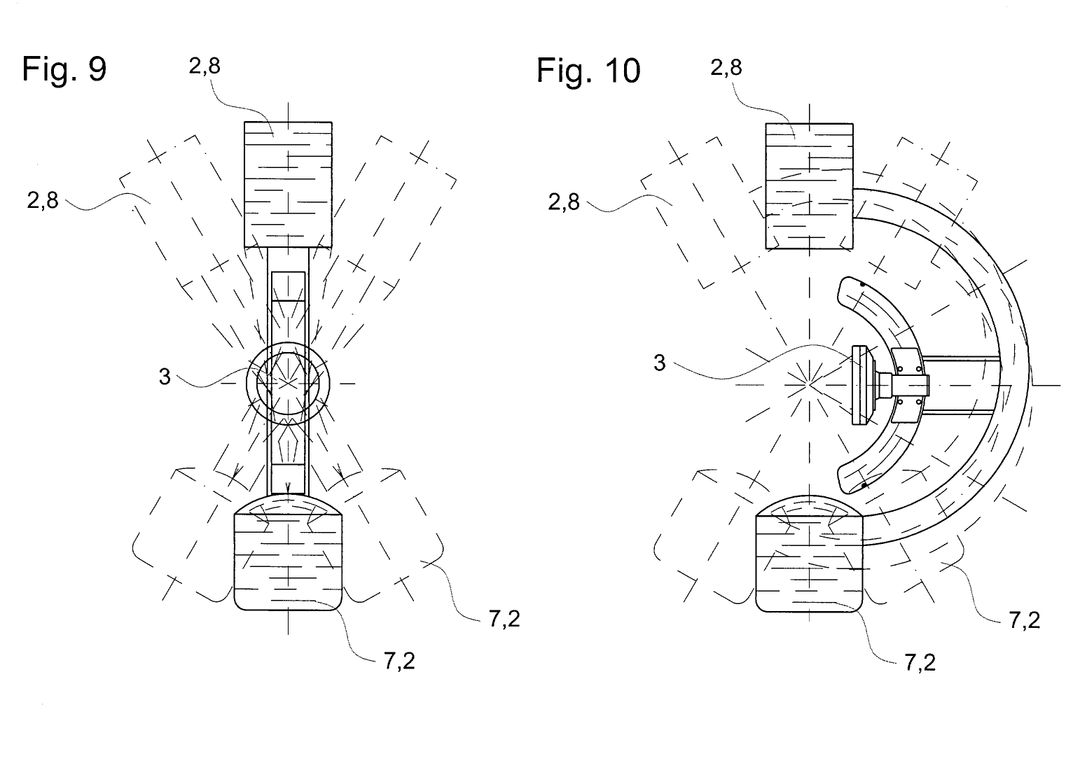

In FIG. 2a the lateral view of the device 1 is to be seen yet again, wherein the position of the therapy soruce 3 is shown in the position which it assumes during the treatment of the patient. FIG. 2b represents the plan view according to section 1-1. As may particularly be seen in FIG. 2b, the guide arm 4 is positioned pivotable about the rotational axis 6. Furthermore the point of rotation 6 is distanced from the middle axis 12 of the X-ray system 2 by a lateral distance s. With this it is made possible for the X-ray system 2 and here specifically the C-shaped connection element 9 to be able to be lateraly adjusted in a manner known per se on treatment (cf. also FIG. 9).

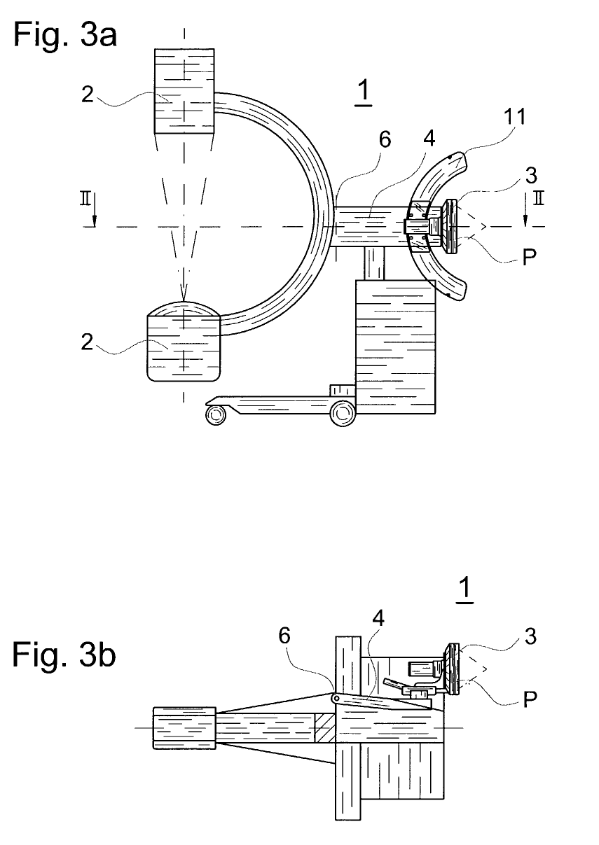

In FIG. 2c apart from the treatment position B of the therapy source 3, as in FIG. 2b.—the so-called parking position P of the therapy source 3 is also sketched. For bringing the therapy source 3 from the treatment position B into the parking position P the guide arm 4 is pivoted about a pivot angle of about 180E. If the therapy source 3 is again to be brought into the treatment position, the corresponding pivoting in opposite directions is carried out. In FIG. 3a and FIG. 3b the parking position P is to be seen in a lateral view and a plan view respectively, however now without the incorporated treatment position.

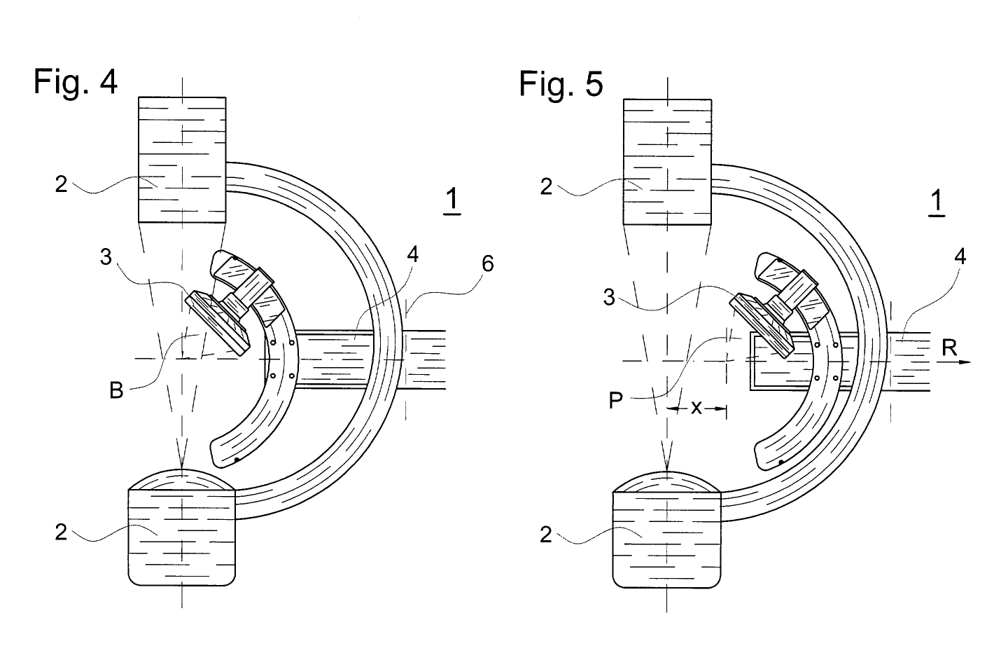

In FIG. 4 the X-ray system 2 and the therapy source 3 in the treatment position B are to be seen in a lateral view. FIG. 5 shows the device in a parking position in which the therapy source 3 at the end of the guide arm 4 is displaced out of the treatment position B by way of the translatory displacement x of the guide arm in the displacement direction R. Already on account of the translatory displacement displacement x a certain accessibility to the patient is made possible. Additionally, as is not shown in FIG. 5, the therapy source 3 may be pivoted about the rotational axis 6 corresponding to FIG. 3a and FIG. 3b.

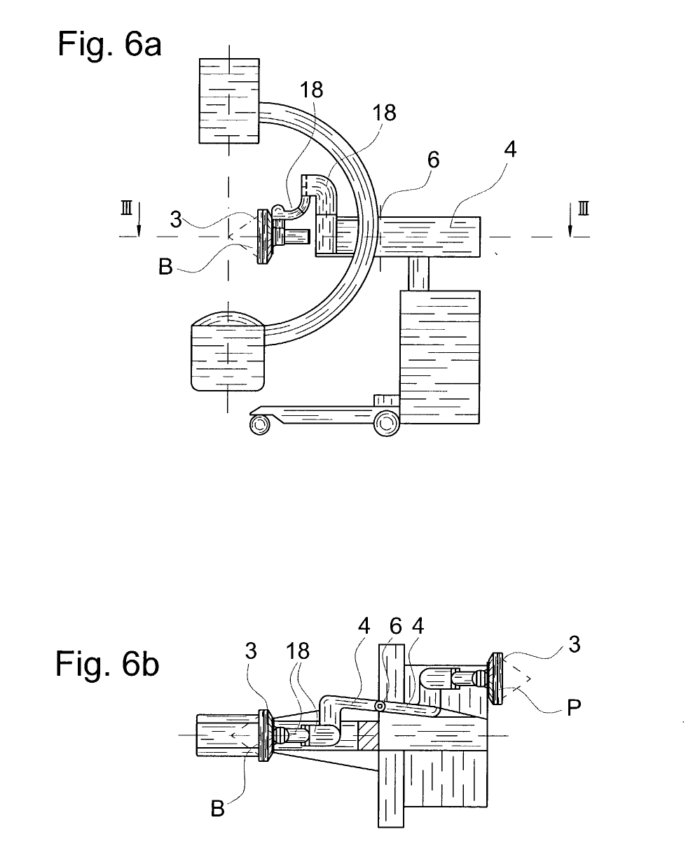

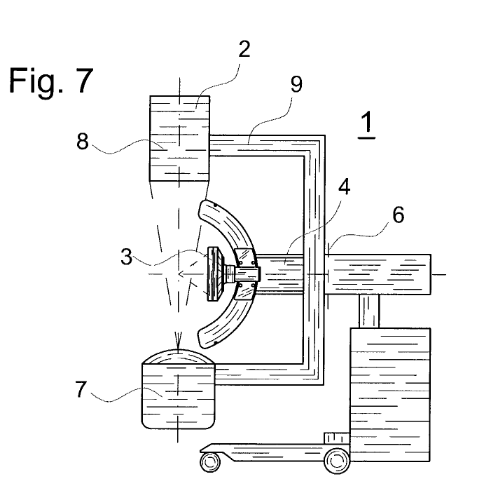

In FIG. 6a and FIG. 6b there is shown a further embodiment form of the therapy apparatus 3. Here one does not apply a pivot rail 11 as with the FIGS. 1 to 5, but a multiple-mounted freely movable holding arm 18 which via suitable sensors (not shown) indicates its position to the X-ray system for the detection of the position. For positioning and aligning the therapy source 3 there are presently envisaged three joints. With this it is possible to exactly align the focus of the therapy source 3 onto the body stone to be destroyed. Otherwise the guide arm 4 is likewise present which by way of translatory displacement and pivoting may move the therapy source 3 quickly out of the treatment position B into the parking position P. In FIG. 6b again both positions B and P are shown. In FIG. 7 it is to be seen that instead of a C-shaped connection element 9 one may also apply a U-shaped element.

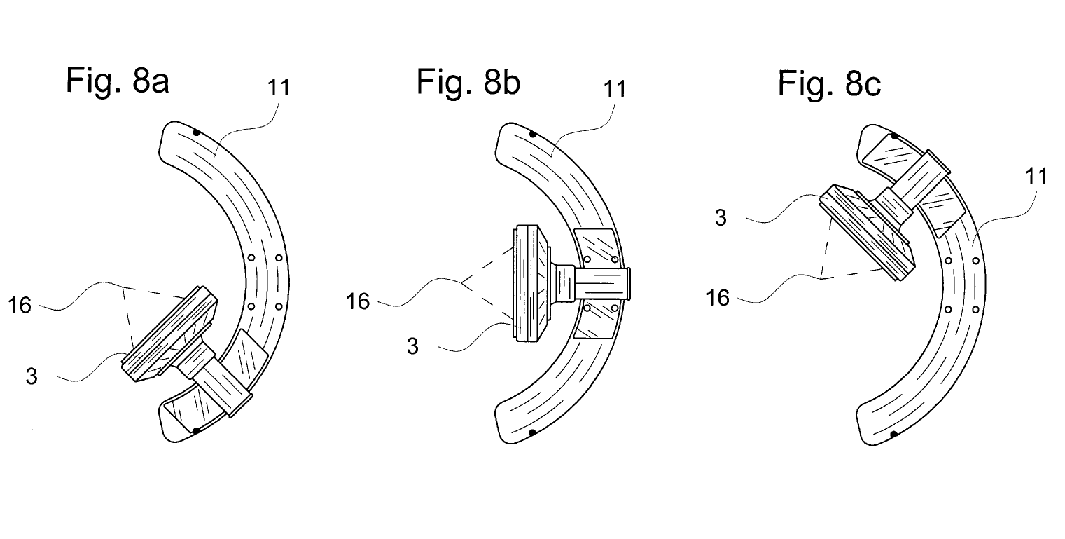

In FIG. 8a, 8b and 8c there are shown three different positions of the therapy source 3 on the pivot rail 11. As is to be seen, the therapy source 3 may be adapted to the given conditions so that the focus 16 is always optimally set to the region of the patient which is to be treated. Presently there are shown a lower, a middle and an upper treatment position.

In the FIGS. 9 and 10 one may finally see that the X-ray system 2 in the 5 known manner may be adjusted laterally (FIG. 9) as well as orbitally (FIG. 10) also with the design suggested according to the invention. The lateral pivoting angle mostly does not exceed 30E.

With all suggested formations it is possible to axially displace the 10 therapy source away for the quick position change of the patient and to additionally be able to completely pivot it out of the irradiation field of the X-ray system into the parking position.

By way of means for measuring or monitoring the translatory displacement x as well as of the pivoting angle it is possible after pivoting the therapy source 3 into the parking position P, by actuating a suitable control to again move the therapy source 3 automatically and exactly again into the desired treatment position B without any form of adjustment becoming necessary. The operating expense for the medical device is accordingly low. Thus one creates the precondition that the X-ray system may be used without problem for applications with which no therapy source is required.

We claim:

1. A medical device with a picture-providing diagnostic X-ray system and with a therapy source, wherein the therapy source is arranged on a guide arm whose one end is translatorily movable relative to the X-ray system into a displacement direction (R), and wherein the guide arm is pivotably arranged about a rotational axis.

2. A device according to claim 1, wherein the displacement direction (R) lies in a horizontal plane and that the rotational axis is arranged vertically.

3. A device according to claim 1, wherein on the guide arm and/or on the X-ray system there are arranged means with which the translatory displacement (x) of the guide arm relative to the X-ray system and the pivoting angle of the therapy apparatus relative to the X-ray system are measurable.

4. A device according to claim 1, wherein the one end of the guide arm is arranged on a translatorily movable carriage.

5. A device according to claim 1, characterised in that between the therapy source and the guide arm, preferably at the end of the guide arm there is arranged a pivot rail.

6. A device according to claim 1, wherein the rotational axis is laterally distanced so far from the middle axis of the X-ray system that the X-ray system is laterally adjustable.
